(19) United States
(12) Patent Application Publication
(10) Pub. No.: US 2022/0133269 A1
(43) Pub. Date: May 5, 2022

(54) INTEGRATED WEARABLE ULTRASONIC PHASED ARRAYS FOR MONITORING

(71) Applicant: The Regents of the University of California, Oakland, CA (US)

(72) Inventors: Sheng Xu, La Jolla, CA (US); Muyang LIN, La Jolla, CA (US); Zhuorui ZHANG, La Jolla, CA (US); Hongjie HU, La Jolla, CA (US); Chonghe WANG, La Jolla, CA (US); Baiyan QI, La Jolla, CA (US)

(21) Appl. No.: 17/431,572

(22) PCT Filed: Feb. 28, 2020
(86) PCT No.: PCT/US2020/020292

§ 371 Date: Aug. 17, 2021

Related U.S. Application Data
(60) Provisional application No. 62/811,770, filed on Feb. 28, 2019.

(51) Int. Cl. A61B 8/00; A61B 8/04; B06B 1/06

(52) U.S. Cl. CPC A61B 8/4427; A61B 8/4488; A61B 8/54; A61B 8/463; H01L 41/0477; A61B 8/04; A61B 8/4472; B06B 1/0629; A61B 8/56

(57) ABSTRACT

Systems and methods are provided that integrate control electronics with a wireless on-board module so that a conformal ultrasound device is a fully functional and self-contained system. Such systems employ integrated control electronics, deep tissue monitoring, wireless communications, and smart machine learning algorithms to analyze data. In particular, a stretchable ultrasonic patch is provided that performs the noted functions. The decoded motion signals may have implications on blood pressure estimation, chronic obstructive pulmonary disease (COPD) diagnosis, heart function evaluation, and many other medical monitoring aspects.

### CROSS REFERENCE TO RELATED APPLICATIONS

### Background

It is known to measure blood pressure in various ways. A standard way is by use of a blood pressure cuff. Alternative and more advanced ways have also been developed.

For example, PCT/US2018/013116 entitled “Stretchable Ultrasonic Transducer Devices” describes a skin-integrated conformal ultrasonic device capable of non-invasively acquiring central blood pressure (CBP). This system requires an ultrasound patch to be wired to a back-end data-acquisition system. While useful, it has the disadvantage of requiring this data coupling.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

Systems and methods according to present principles meet the needs of the above in several ways.

In particular, there is a need for integration of control electronics with a wireless on-board module so that a conformal ultrasound device is a fully functional and self-contained system. Such provides an important step in the translation of this system from the bench-top to the bedside. Such systems may employ integrated control electronics, deep tissue monitoring, wireless communications, and smart machine learning algorithms to analyze data.

In one aspect, methods, devices and systems are disclosed that pertain to a fully integrated smart wearable ultrasonic system. Such systems and methods allow for human bio-interface motion monitoring via a stretchable ultrasonic patch. The decoded motion signals may have implications on blood pressure estimation, chronic obstructive pulmonary disease (COPD) diagnosis, heart function evaluation, and many other medical monitoring aspects.

In one aspect, the invention is directed toward a system for monitoring a physiologic parameter, including: a conformal ultrasonic transducer array coupled to a flexible substrate; an analog front end circuit coupled to the flexible substrate and further coupled to the conformal ultrasonic transducer array, the analog front end circuit configured to generate ultrasonic acoustic waves and receive reflected ultrasonic acoustic waves; a digital circuit coupled to the flexible substrate and further coupled to the analog front end circuit, the digital circuit configured to at least: control the analog front end circuit at least in its generation of ultrasonic acoustic waves; transmit an indication of the received reflected ultrasonic acoustic waves to an external computing environment.

Implementations of the invention may include one or more of the following. The system may further include the external computing environment, and the external computing environment may be configured to generate and display an indication of the monitored organ function. The external computing environment may also be configured to measure a shift, the shift in the time domain, in a detected peak of the received reflected acoustic wave, the shift due to movement of an organ or tissue, and the displayed indication of the monitored physiologic parameter may be based on the measured shift. Recognition of the shift may be based at least in part on a step of machine learning. The displayed indication may be based on a step of machine learning, the machine learning associating the shift with the monitored physiologic parameter. The analog front end may be further configured to steer or direct the generated ultrasonic acoustic waves toward an organ, tissue, or location of interest, the steering or directing by beamforming. The steering may include dynamically adjusting a time-delay profile of individual transducer activation in the transducer array, which may include a piezoelectric array. The flexible substrate may be made of polyimide. The monitored physiologic parameter may be central blood pressure or COPD.

In another aspect, the invention is directed toward a method for monitoring a physiologic parameter, including: determining a location of interest, the location associated with the physiologic parameter to be monitored; transmitting ultrasonic acoustic waves toward the location of interest; receiving reflected ultrasonic acoustic waves from the location of interest; transmitting an indication of the received reflected ultrasonic acoustic waves to an external computing environment; receiving the received reflected ultrasonic acoustic waves at the external computing environment; detecting a shift in the time domain of the received reflected ultrasonic acoustic wave; determining an indication of the monitored physiologic parameter based at least in part on the shift; and displaying the indication of the monitored physiologic parameter; where at least the transmitting and receiving reflected ultrasonic acoustic waves, and the transmitting an indication, are performed by components within an integrated wearable device.

Implementations of the invention may include one or more of the following. The monitored physiologic parameter may be central blood pressure. The transmitting ultrasonic acoustic waves toward the location of interest may include a step of steering the ultrasonic acoustic waves toward the location of interest, where the steering includes dynamically adjusting a time-delay profile of individual transducer activation in the transducer array. The and receiving ultrasonic acoustic waves may be performed at least in part by a piezo-electric array. The detecting a shift of the received reflected ultrasonic acoustic wave, the shift in a peak in the time domain, may include a step of recognizing the shift using machine learning. The determining an indication of the monitored physiologic parameter may be based at least in part on the shift and may include a step of associating the shift with the physiologic parameter using machine learning. The machine learning may be learned on a training set of ultrasound data.

Advantages of the invention may include, in certain embodiments, one or more of the following. The biomedical imaging claimed here are those visible by ultrasound, including but not confining to blood vessel walls, diaphragm, heart valves, etc. Compared with the existing ultrasound imaging probe, in one aspect, this new ultrasonic imaging system overcomes the challenge of locating uncertain positions of the transducers using an unsupervised machine-learning algorithm. Furthermore, this technology may also perform a real-time artificial intelligence (AI) analysis to extract hemodynamic factors like blood pressure, blood flow, and cardiac pressure signals from ultrasound images. Other advantages will be understood from the description that follows, including the figures and claims.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1 shows a schematic of an implementation according to present principles.

FIG. 2A shows a more detailed schematic of an implementation according to present principles.

FIG. 2B shows a more detailed implementation of an analog front end according to present principles.

FIG. 3 shows a more detailed implementation of an exemplary transducer unit according to present principles.

FIG. 4 shows an exemplary hardware design for a wireless ultrasound front end (circuit schematic) according to present principles.

FIG. 5 illustrates time control logic of the MCU to realize pulse generation, RF signal digitization, and data transmission, in one pulse repetition interval.

FIG. 6A illustrates GUI schematics of software in the automated signal processing algorithm workflow, using blood vessel distention monitoring as an example.

FIG. 6B shows steps in automatic channel selection and automatic motion tracking.

FIG. 6C shows exemplary software design for autonomous artery recognition and wall tracking.

FIG. 7 shows an example of peak shifting.

FIG. 8A shows use of an unsupervised machine-learning algorithm to find transducer locations to enhance the quality of the reconstructed images.

FIG. 8B shows a proposed algorithm for ultrasound image quality enhancement.

FIG. 8C shows schematically enhancement of images.

FIG. 9 illustrates deep learning architectures (9A) and bidirectional domain adaptation methods (9B) used for ultrasound image interpretation.

FIGS. 10A and 10B illustrate use of the conformal ultrasound patch on a user. FIG. 10B also illustrates the central vessels in the human neck.

FIG. 11 illustrates an exemplary implementation of a conformal ultrasonic transducer array indicating conformance to a curved body surface.

FIG. 12-15 illustrates an exemplary implementation of a system and method according to present principles, in particular arranged as a densely arrayed device for imaging and Doppler ultrasound.

FIG. 12 illustrates a core technique for receiving beamforming.

FIGS. 13A and 13B illustrate an application of the technique according to present principles, employed in non-destructive testing.

FIG. 14 illustrates an application of the technique according to present principles, employed in B-mode ultrasound.

FIG. 15 illustrates a core technique for transmission beamforming.

FIGS. 16A and 16B illustrate an application of the technique according to present principles, employed in tissue Doppler imaging.

FIGS. 17A and 17B illustrates an application of the technique according to present principles, employed in blood flow monitoring.

Like reference numerals refer to like elements throughout. Elements are not to scale unless otherwise noted.

### DETAILED DESCRIPTION

Arrangements according to present principles include materials, devices, systems and methods that pertain to a fully integrated smart wearable ultrasonic system. Depending on implementation, the following functional modules may be employed.

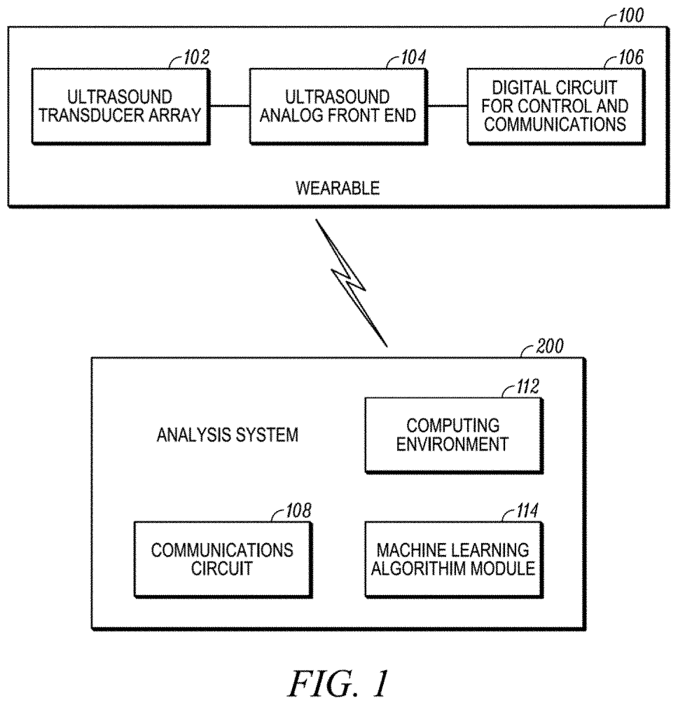

Referring to FIG. 1, a wearable 100 may include an ultrasound transducer array 102 coupled to an ultrasound analog front end (AFE) 104 and a digital circuit for control and communications 106. The wearable 100 may be coupled to a receiver 200 that includes an analysis system including a communications circuit 108 for reception of signals from digital circuit 106. The receiver 200 further includes a computing environment 112 running interactive software that may be in communication with various back-end devices, e.g., smart phones, to allow visualization of the human bio interface motion waveforms. The machine learning algorithm module 114 may also be employed for various functionality, including automatic transducer channel selection and interface motion waveform decoding from ultrasonic RF signals.

The ultrasound transducer array 102 may be a conformal array delivering the ultrasound as well as receiving reflected acoustic signals. The ultrasound analog front end 104 may be employed for ultrasound generation, echo signal receiving, and amplification. Other components of the AFE include high-voltage pulsers, transmit/receive (T/R) switches, multiplexes, and radio frequency (RF) amplifiers.

The digital circuit 106 may be employed for system control, signal digitalization, onboard transmission, and high-speed wireless transmission, and other functionality as may be required. Such a digital circuit 106 generally includes a microcontroller unit (MCU) with built-in analog to digital converters (ADC) as well as Wi-Fi modules.

Various aspects of these modules will now be described in more detail, as well as the use of the same in the noninvasive measurement of central blood pressure and other applications.

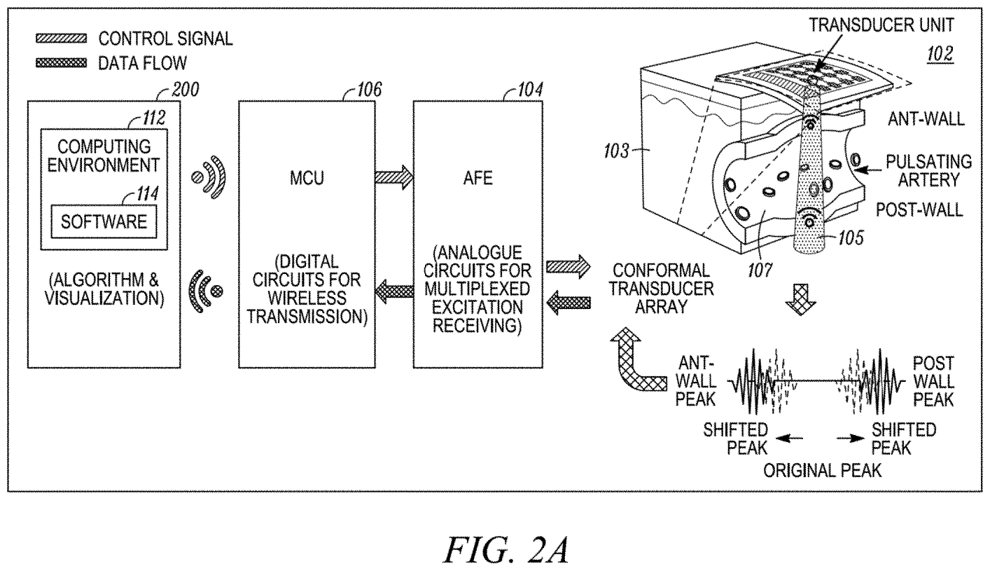

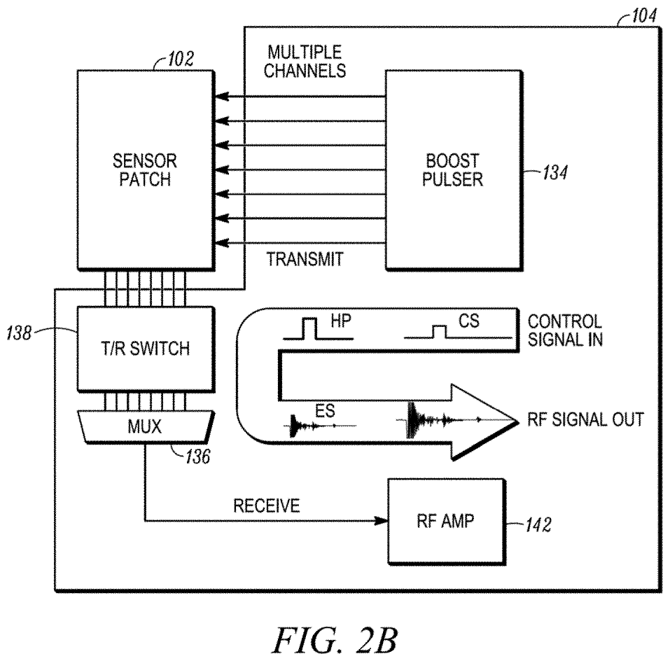

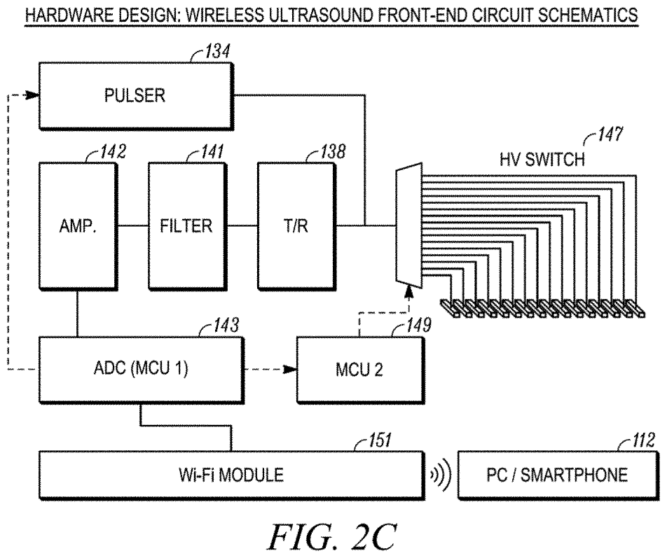

The general principle of bio-interface motion monitoring is illustrated in FIG. 2A, which illustrates a device tracking blood vessel wall motion. The ultrasound transducer element 102 above the target bio-interface A (103) generates ultrasound 105 and receives the reflected signals from it. As may be seen, the acoustic waves being transmitted by the transducer unit may be aimed and targeted at a particular element, e.g., a pulsating artery 107.

When these interfaces move, the reflected peaks shift in the time domain corresponding to their motion. All the signals are amplified through the AFE 104, digitalized by ADCs in the MCU within digital circuit 106, and wirelessly transmitted to a smartphone or other analysis system 200, which may run software 114. A machine learning algorithm incorporated in the software 114 may be employed to recognize the reflected signals of the target interfaces and capture their movement trajectory continuously. The algorithm may be situated on the smartphone or on, e.g., a connected computing environment such as a cloud server. The algorithm may employ machine learning to recognize the shifts caused by the motion of the location of interest and may further use machine learning to associate the shifts with parameters desired to be monitored, e.g., physiologic parameters desired to be determined for diagnosis and other purposes.

In more detail, in a first step, and referring to FIGS. 2B and 2C, the analog front-end circuit 104, coupled to the transducer array 102, includes a multiplexer 136, high-voltage boost pulsers 134, radio frequency (RF) amplifier 142, transmit/receive (T/R) switches 138, and an analog-to-digital-converter. Multiple channels allow for beam steering and the same emerge from a boost pulser 134 which is controlled by the digital circuit 106 to generate ultrasound. Echo signals are enlarged and collected using a T/R switch 138 and demultiplexer 136 and amplifier 142, which form part of the high-speed analog-to-digital-converter. An inset shows the flow of signals.

Second, the digitalized signals are processed by a field-programmable-gate-array (FPGA) or an MCU. Raw ultrasound data may be decoded into the blood pressure waveforms. Finally, the decoded waveforms may be wirelessly transmitted and visualized on a display via Bluetooth or Wi-Fi. A rechargeable miniaturized battery may provide the power for the entire system.

The ultrasound transmitter is made by a boost circuit which transforms a low-voltage control signal (CS) to a high-voltage pulse. The T/R switches are used to cut off over-ranged voltages and protect the receiving circuit. Multiplexers are used for channel selection. RF amplifiers amplify the received echo signals (ES) for the following ADC sampling. All the components may be fabricated on a flexible printed circuit board (FPCB).

FIG. 2C illustrates another implementation of a wireless ultrasound front-end circuit with similar components in a similar arrangement.

As may be seen, the hardware that interfaces with the soft ultrasonic probe may perform transducer selection, transducer activation, echo signal receiving, and wireless data transmission. In one implementation, the high-voltage (HV) switch 147 controlled by a microcontroller (MCU) 149 may select a proper number of transducers as active pixels. Once the active pixels are selected, the pulser 134 may deliver electrical impulses to the pixels to generate the ultrasound wave. After the ultrasound is generated, the echo signal receiving may start. The received signal may pass the transmit/receive (T/R) switch 138 and the analog filter 141 to be amplified by the RF amplifier 142. Finally, the amplified signal may be received by the analog-to-digital converter (ADC) 143, which may also be an MCU. Once the signal is received and digitalized, the Wi-Fi module 151 may transmit the signals wirelessly to terminal devices (e.g., PC or smartphone) 112.

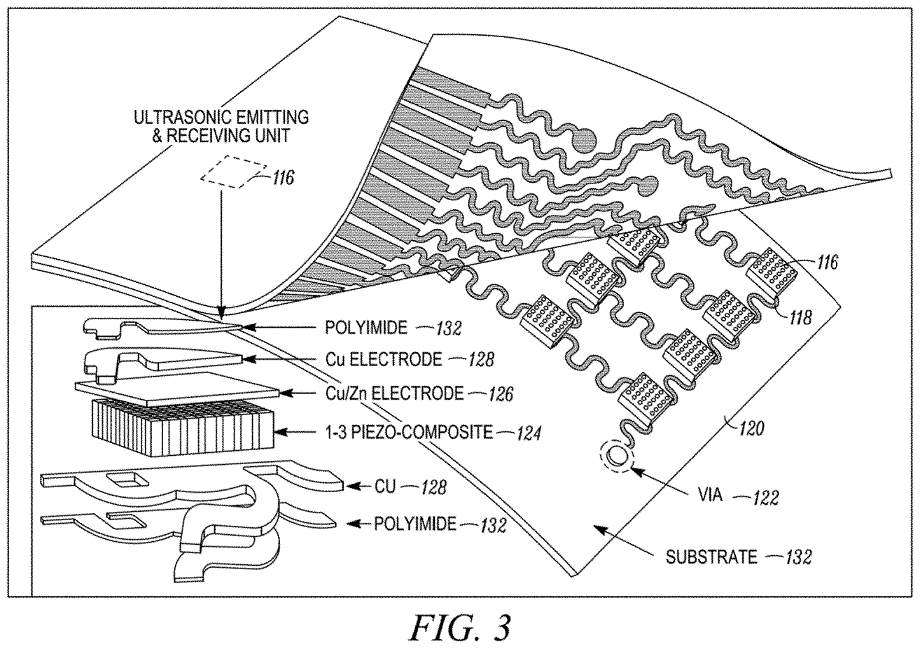

Details of an exemplary conformal ultrasonic transducer array are shown in FIG. 3, which illustrates a schematic of a conformal ultrasonic transducer array and the structure of a single transducer element (inset). In this exemplary embodiment, an “island-bridge” structure is used to provide the device with sufficient flexibility to provide suitable conformity to the skin.

Rigid components 116 are integrated with the islands, and the wavy serpentine metal interconnects 118 serve as the bridges. The bridges can bend and twist to absorb externally applied strain. Therefore, the entire structure is rigid locally in the islands, but stretchable globally by adjusting the spacing between the rigid islands during the bending, stretching, and twisting processes. The result is a natural interface that is capable of accommodating skin surface geometry and motions with minimal mechanical constraints, thereby establishing a robust, non-irritating device/skin contact that bridges the gap between traditional rigid planar high-performance electronics and soft curvilinear dynamic biological objects. In one implementation, the ultrasound transducers, which are the rigid components 116, are provided on a substrate 120 having a via 122 for interconnects.

As seen in the inset, an exemplary element 116 may employ a 1-3 piezo-composite ultrasound array component 124, also known as piezo pillars, covered by a Cu/Zn electrode 126, which is covered by a Cu electrode 128 on both top and bottom sides, and with a polyimide covering 132. However, it should be noted that active ultrasonic materials used here are not confined to 1-3 composites but may employ any rigid piezoelectric materials. The polyamide layers may provide the substrate as well as the cover.

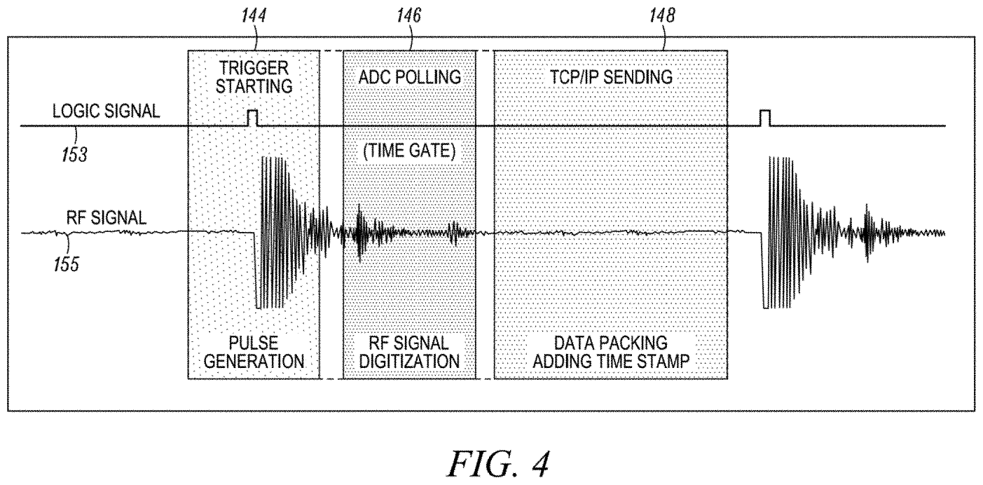

FIG. 4 illustrates the working logic of the digital circuit 106. As noted above, the digital circuit may include an MCU 149, integrated ADCs, e.g., elements 143, and a Wi-Fi module 151. Referring now to the figure, for ultrasound transmission, a triggering signal 153 is used for ultrasound pulse generation in a triggering step 144. Following this triggering signal 153, the RF signal 155 of the ultrasound echo received by the transducer. Simultaneously ADCs are activated for the digital sampling of the received ultrasonic echo in step 146. To realize a sufficient sampling frequency, the embedded ADCs may in one implementation work in an interleaved manner. The designed sampling rate may be proportional to the number of embedded ADCs and the sampling rate of one. A typical synthetic sampling rate is 20 MHz. ADCs may work through a predefined time gate range and store all the data into the built-in memory of MCU. After that, this data may be transmitted wirelessly to the terminal device through TCP/IP protocols in step 148. Direct memory access (DMA) techniques may be employed to guarantee data access speed. This digital circuit may be fabricated on an FPCB platform and integrated into the AFE circuit.

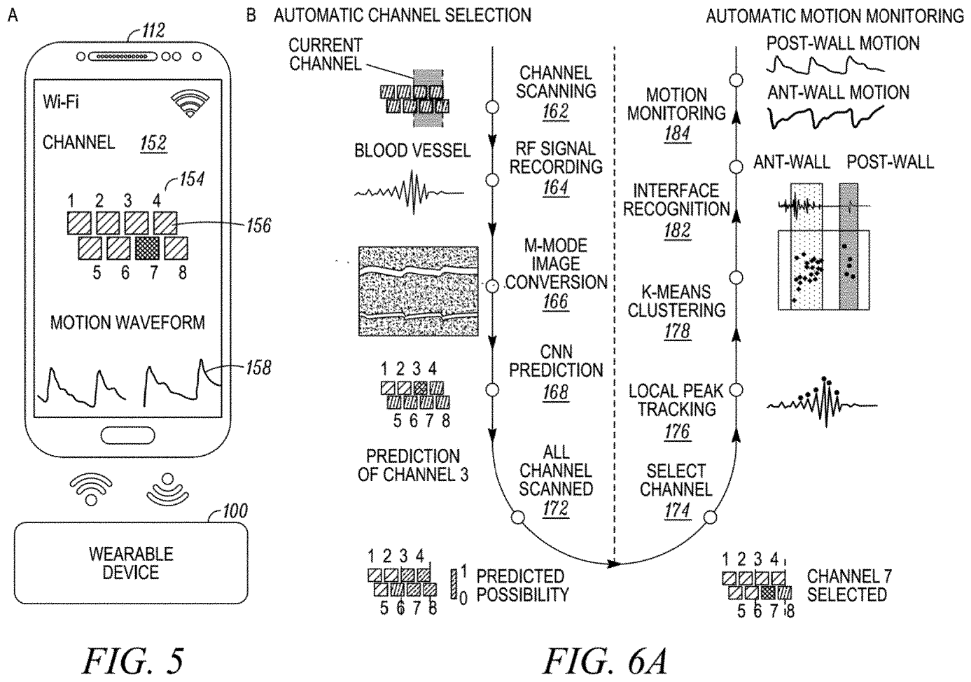

Referring to FIG. 5, software 152 may be employed on the terminal device 112, e.g., a computing environment such as a smartphone, laptop, tablet, desktop, or the like, to receive the wirelessly transmitted data from the wearable device 100, to process the data, and to visualize the detected bio-interface motion (e.g., motion of arterial walls). For example, on a graphical user interface (GUI) 154, the user can connect the back-end terminal 112 to the wearable device 100. Channel selection 156 can be either done manually by the user or automatically. The motion waveform 158 can be viewed through the terminal device, e.g., a suitable computing environment.

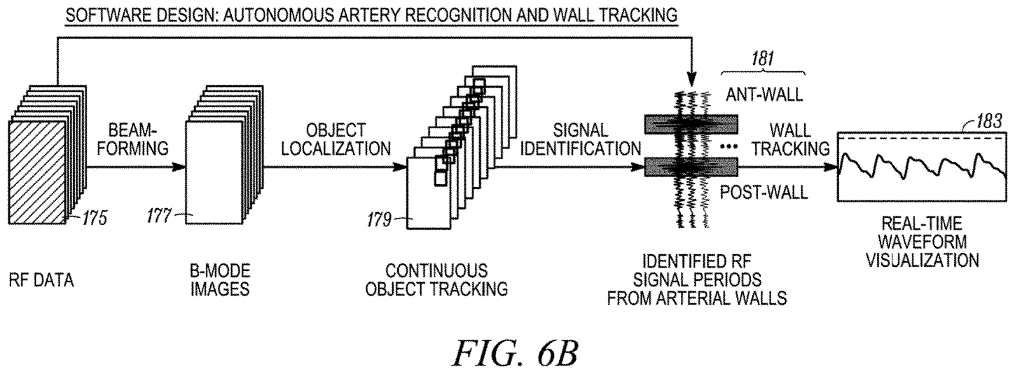

Algorithms may then be employed using machine learning for automated signal processing. In particular, and referring to FIG. 6A, machine learning algorithms may be employed to achieve at least the following two major functionalities: automatic channel selection and bio-interface motion tracking.

Referring to the steps shown in FIG. 6A, for channel selection, RF signals may be scanned 162 and may be recorded 164 for a certain channel, and the same may then be transformed 166 to an M-mode image. This image may be input to a developed convolutional neural network (CNN) model. A predicted possibility of “this channel is at the correct position”, may be assessed 168. After scanning all the channels 172, a most possible channel may be determined or selected 174 and used for bio-interface motion monitoring. Peaks may be tracked 176 and a K-means clustering algorithm 178 may be used to recognize 182 which part of the signal represents the target bio-interface. Finally, the motion of the target may be tracked by, e.g. Kalman filters, applied 184 to the recognized signal regions.

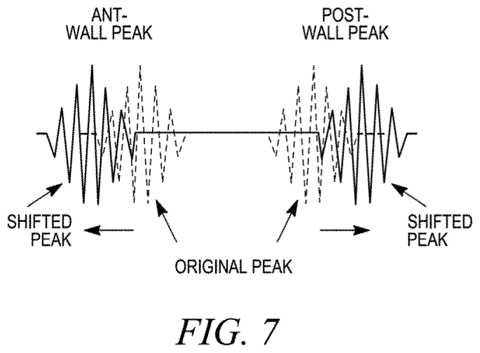

Referring to FIG. 6B, an illustration may be seen of software design according to present principles, including autonomous artery recognition and wall tracking. The ultrasound RF data 175 results in B-mode images 177 from which objects may be localized. This functionality may be achieved by various deep learning models that are designed for object localization. By detecting the object through a series of successive frames, continuous object tracking 179 may be performed, and, e.g., wall tracking 181 using shifted signals (see FIG. 7) may be performed through cross-correlation of the original RF signals. Finally, the processed carotid wall waveforms 183 may subsequently be visualized on the graphical user interface.

As noted above, when the interfaces move, the reflected peaks will shift in the time domain corresponding to their motion. This may be seen in FIG. 7, in which the original peaks of an anterior wall and a posterior wall are shown shifted.

The whole system may integrate at least two major functional modules: ultrasound image enhancement, finding the transducer locations and thereby enhancing the quality of the reconstructed images, and ultrasound image analysis, which automatically analyzes the ultrasound images acquired from the soft ultrasound probe.

Regarding the first major functional module, a major challenge of using soft probes to perform ultrasound imaging is that the locations of transducer elements are uncertain for most application scenarios. For proper image reconstruction, transducer element locations should be determined at sub-wavelength level accuracy. In conventional ultrasound probes for diagnosis purposes, the transducers are fixed in a planar surface through a rigid housing. However, when integrated onto the human skin, the soft probe is on and conforms to dynamic curvilinear surfaces and the transducer locations will be ever-changing. Therefore, images reconstructed from the soft probe will be significantly distorted if no proper method is applied to compensate for the transducer element displacement.

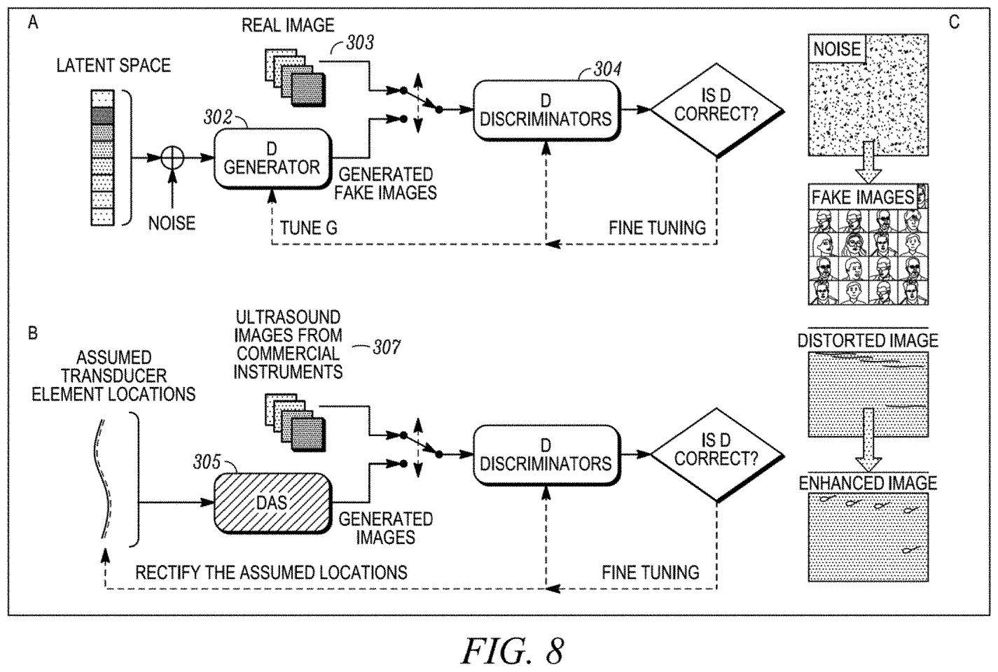

To solve this problem, an unsupervised machine-learning algorithm may be applied to find the transducer locations and thereby enhance the quality of the reconstructed images. The algorithm is inspired by a generative adversarial network (GAN), shown in FIG. 8A. FIG. 8A shows working principles and applications of a conventional GAN and in FIG. 8B a proposed algorithm for ultrasound image quality enhancement is illustrated. GANs consist of a generator 302 and a discriminator 304. The generator 302 (G) synthesizes images while the discriminator 304 (D) attempts to distinguish these from a set of real images 303. The two modules are jointly trained so that D can only achieve random guessing performance. This means that the images synthesized by G are indistinguishable from the real ones. In the proposed solution, shown in FIG. 8B, the GAN generator is replaced by a standard delay-and-sum (DAS) algorithm 305 for ultrasound image reconstruction. The two modules may be trained using a large dataset of ultrasound images 307 from commercial instruments as the training set of real images. The algorithm takes the radiofrequency voltage data acquired from the soft probe as input and learns the DAS beamformer parameters needed to reconstruct the ultrasound images. The training proceeds until these reconstructed images cannot be distinguished from the existing real images.

Regarding ultrasound image analysis, a neural network-based model is developed to automatically analyze the ultrasound images acquired from the soft ultrasound probe. The blood pressure, blood flow, and cardiac pressure signals can be extracted from ultrasound images (M-Mode 403, Doppler 405, and B-mode 407, respectively) using deep learning networks trained for semantic segmentation. Conventionally, this model works well after training from large image datasets. However, such datasets are not likely to be available, at least initially, for a soft-probe ultrasound. To overcome this problem, two sets of techniques are applied to enable training with small datasets.

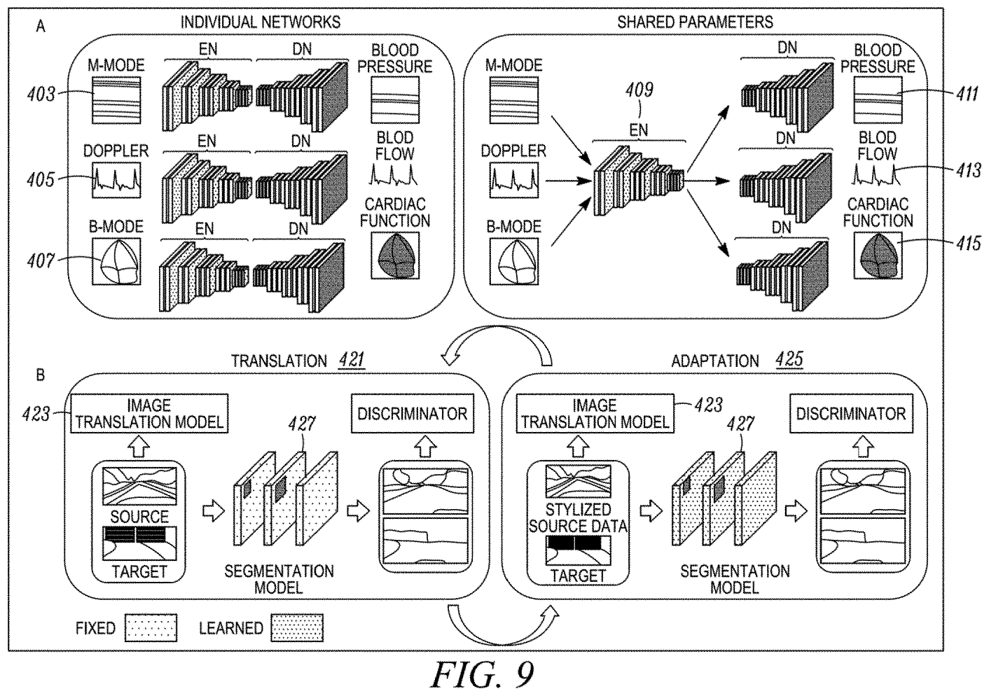

In more detail, FIG. 9 illustrates deep learning architectures (9A) and bidirectional domain adaptation methods (9B) used for ultrasound image interpretation. Note that “EN” indicates an encoder network and “DN” indicates a decoder network.

The first technique for enabling training with small datasets, illustrated in FIG. 9A, relies on parameter sharing between the different tasks. This leverages the fact that modern segmentation networks are implemented with an encoder-decoder pair. The encoder abstracts the input image into a lower-dimensional code that captures its semantic composition. The decoder then maps this code into a pixel-wise segmentation. Usually, a network would be learned independently per task. This, however, requires learning a large number of parameters. The architectures in this AI system include those shown on the right in FIG. 9A, where the parameters are shared across tasks. In particular, the encoder 409 is shared through the three tasks (411 and 413 and 415). Therefore, the overall number of parameters to learn is reduced and suitable for training on small datasets.

The second, illustrated in FIG. 9B, relies on image transfer techniques. The goal is to leverage existing large ultrasound datasets to help train the networks of FIG. 9A. The architecture here is the domain adaptation. The domain adaptation applies a network trained on a large dataset of images (in this case, existing ultrasound images), known as the source domain, to a new target domain (in this case, soft-probe ultrasound images) where large datasets do not exist. This usually exceeds the performance of a network trained on the target domain. In this system, the bidirectional adaptation is used to keep the performance of the network. This iterates between two steps. In the translation step 421, an image to image translation model 423 is used to translate images of existing ultrasound into images of soft-probe ultrasound. In the adaptation step 425, an adversarial learning procedure is used to transfer the segmentation model 427 trained on the former to the latter. The procedure iterates between the two steps, gradually adapting the network learned on the soft-probe ultrasound. This algorithm is applied to the architectures of FIG. 9A, to further increase the robustness of the segmentation.

### Example: Central Blood Pressure Monitoring

In an exemplary embodiment, systems and methods may be applied to a skin-integrated conformal ultrasonic device 502 for non-invasively acquiring central blood pressure (CBP) waveforms from deeply embedded vessels.

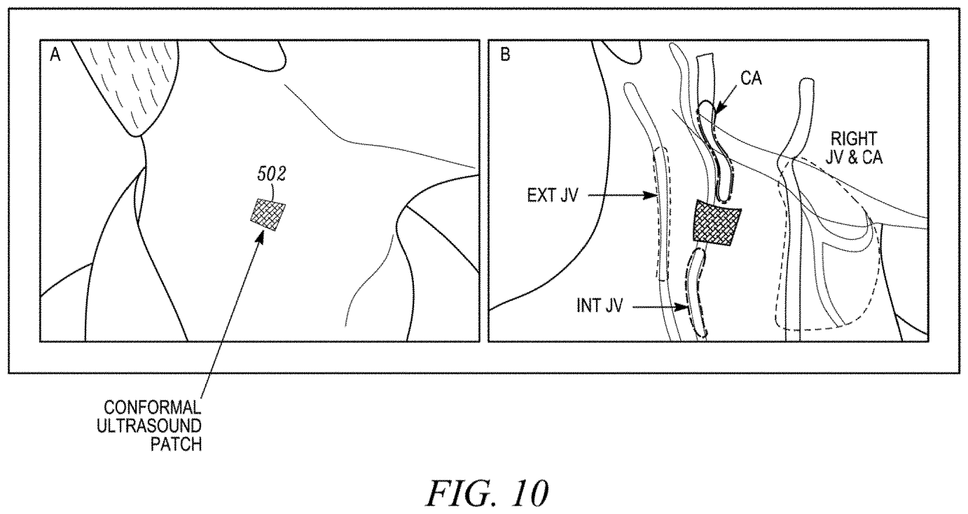

FIGS. 10A and 10B illustrate the use of the conformal ultrasound patch on a user. When mounted on a patient's neck, the device allows the monitoring of the CBP waveform by emitting ultrasound pulses into the deep vessel. FIG. 10B illustrates the central vessels in the human neck. CA is the carotid artery, which connects to the left heart. JV is the jugular vein which connects to the right heart. Both arteries lie approximately 3-4 cm below the skin.

Due to its proximity to the heart, CBP can provide a better, more accurate way to diagnose and predict cardiovascular events than measuring peripheral blood pressure using a cuff. The conformal ultrasound patch can emit ultrasound that penetrates as far as ˜10 cm into the human body and measure the pulse-wave velocities in the central vessels, which can be translated into CBP signals from near the heart.

Additionally, a blood pressure cuff can only determine two discrete blood pressure values, systolic and diastolic. However, blood pressure levels are dynamic at every minute, fluctuating with our emotions, arousal, meals, medicine, and exercise. The cuff can therefore only capture a snapshot of an episode. As the conformal ultrasound patch can emit as many as 5000 ultrasound pulses per second when continuously worn on the skin, it thus offers a continuous beat-to-beat blood pressure waveform. Each feature in the waveform, e.g., valleys, notches, and peaks, corresponds to a particular process in the central cardiovascular system, providing abundant critical information to clinicians.

As indicated above and as will be described in greater detail below, the patch's control electronics are able to focus and steer the ultrasound beam to accurately locate the target vessel, regardless of the patch's location and orientation, so that any user-errors may be corrected automatically. An integrated Bluetooth antenna may wirelessly stream the blood pressure waveform to the cloud for further analysis.

In current clinical practice, CBP is only accessible by implanting a catheter featuring miniaturized pressure sensors into the vessel of interest. This type of measurement, often done in the operating room and intensive care unit, which is significantly invasive and costly and does not allow routine and frequent measurements for the general population. Systems and methods according to present principles, using the conformal ultrasound patch described, leads to not only improving the diagnosis outcome and patient experience, but also empowering the patient with the capability to continuously self-monitor their blood pressure anywhere and at any time. The large amount of data acquired may provide the basis for analyzing blood pressure fluctuation patterns, which is critical for precisely diagnosing and preventing cardiovascular disease.

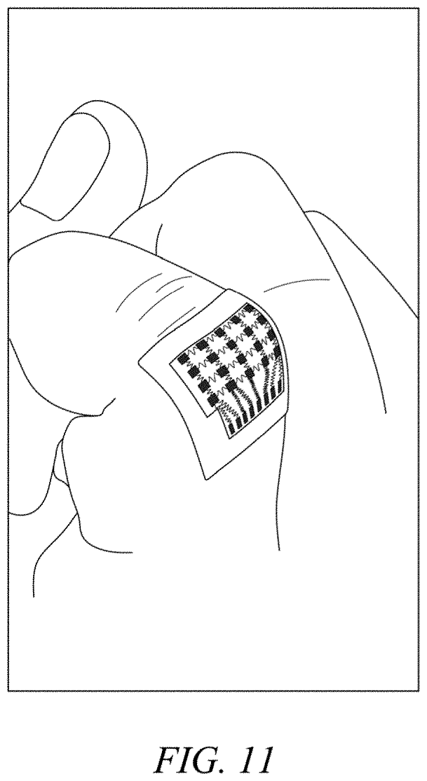

FIG. 11 illustrates an exemplary implementation of a conformal ultrasonic transducer array indicating conformance to a curved body surface.

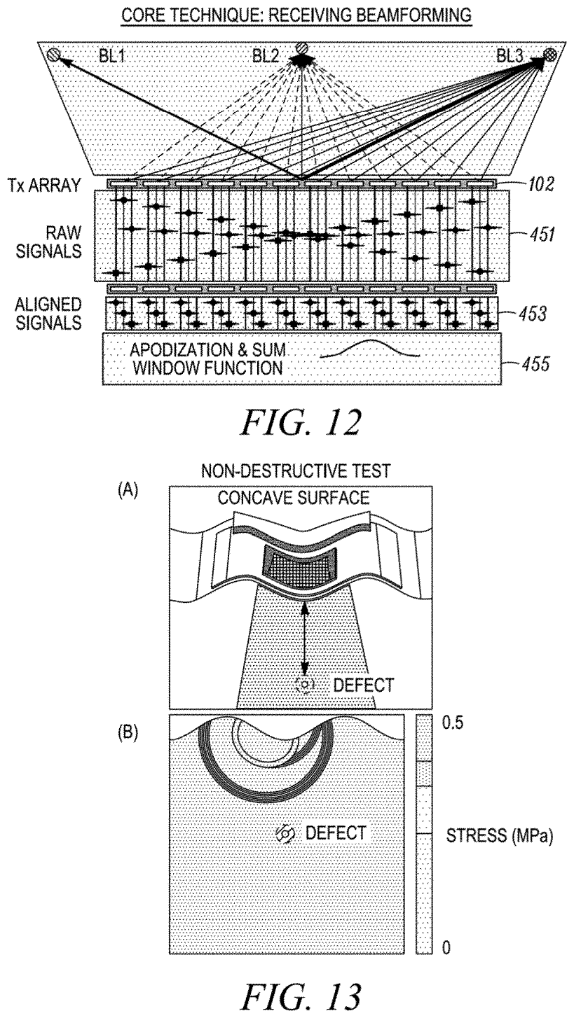

FIG. 12-15 illustrates an exemplary implementation of a system and method according to present principles, in particular arranged as a densely arrayed device for imaging and Doppler ultrasound. In FIG. 12, the transducer array 102 receives the reflected beam. To construct high-resolution ultrasound images, densely arrayed transducers are often used. However, the dense arrangement of transducers sacrifices the transducer size. Thus, each fine transducer element 116 within array 102 will have a weaker signal amplitude compared with a large transducer.

To address this challenge, receiving beamforming technology is developed. The ultrasound signals received by each fine element 116 are added up according to the phase delay between channels to increase the signal-to-noise ratio. In other words, the raw signals 451 are aligned so as to create aligned signals 453. Furthermore, the receiving apodization, which is using window functions to weight the received signals (collectively referred to as step and/or module 455), may be employed to further enhance the image contrast.

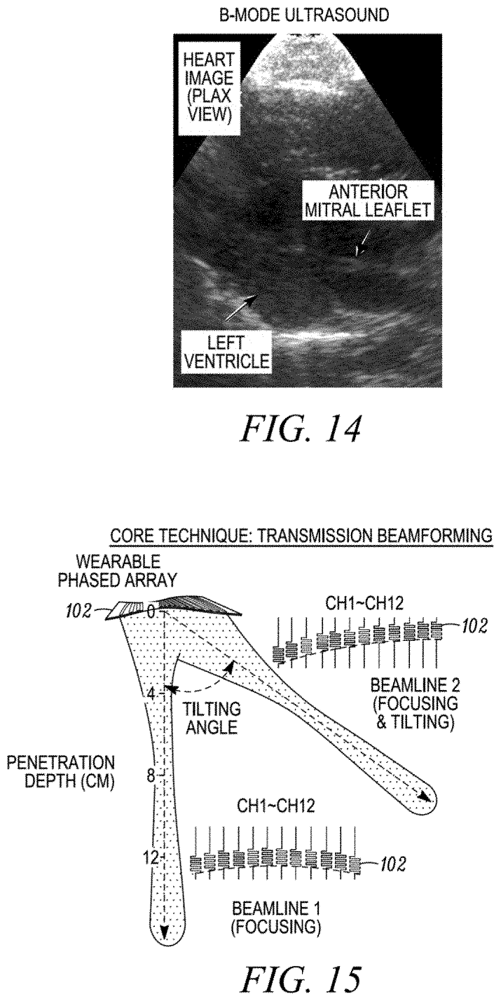

Leveraging this beamforming technology, non-destructive tests on both metal workpieces and biomedical B-mode image could be achieved with the stretchable ultrasound patches as shown in the example applications and as indicated in FIGS. 13A/13B and FIG. 14, respectively.

### Transmission Beamforming

Unlike traditional rigid ultrasound probes, which could easily create any desired Doppler angle by probe manipulation, a stretchable ultrasound patch cannot be physically tilted to create a proper incident angle for Doppler measurement.

However, by leveraging transmission beamforming technology, the ultrasound beam can be tilted and focused electronically. To achieve beam tilting and focusing at the target point, especially on dynamic and complex curvature, an active and real-time time-delay profile can be automatically calculated and applied to each transducer element. Specifically, real-time and high-speed phase aberration method may be adopted to realize this task. One primary principle of the phase aberration correction is that the received signal in one channel can be approximated by a time-delayed replica of the signal received by another channel. Therefore, time-of-flight errors (i.e., phase aberrations) can be found as the position of the maximum in the cross-correlation function. In this way, the phased delay can be calculated to compensate for the error brought by the displacement of each element. The emitted beam of every element will interfere with each other and thus synthesize a highly directionally steered ultrasound beam. The ultrasonic beam can be tilted in a wide transverse window (from −20° to 20°) by tuning the determined time-delay profile. The steerable ultrasonic beam allows the creation of appropriate Doppler angles at specific organs/tissues of interest in the human body.

Examples below show the continuous monitoring of the contractility of the myocardium tissue and blood flow spectrum in the carotid artery respectively.

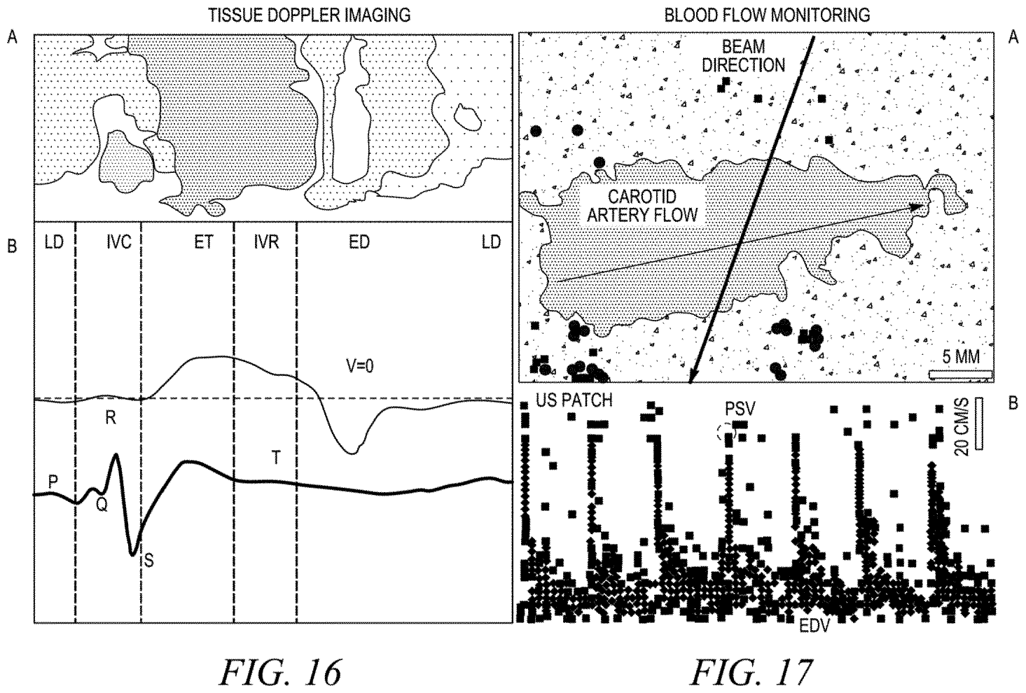

In particular, FIGS. 16A and 16B illustrate an application of the technique according to present principles, employed in tissue Doppler imaging of myocardium tissue, and FIGS. 17A and 17B illustrate an application of the technique according to present principles, employed in blood flow monitoring specifically of the carotid artery.

The system and method may be fully implemented in any number of computing devices. Typically, instructions are laid out on computer-readable media, generally non-transitory, and these instructions are sufficient to allow a processor in the computing device to implement the method of the invention. The computer-readable medium may be a hard drive or solid-state storage having instructions that, when run, are loaded into random access memory. Inputs to the application, e.g., from the plurality of users or from any one user, may be by any number of appropriate computer input devices. For example, users may employ a keyboard, mouse, touchscreen, joystick, trackpad, other pointing device, or any other such computer input device to input data relevant to the calculations. Data may also be input by way of an inserted memory chip, hard drive, flash drives, flash memory, optical media, magnetic media, or any other type of file—storing medium. The outputs may be delivered to a user by way of a video graphics card or integrated graphics chipset coupled to a display that maybe seen by a user. Alternatively, a printer may be employed to output hard copies of the results. Given this teaching, any number of other tangible outputs will also be understood to be contemplated by the invention. For example, outputs may be stored on a memory chip, hard drive, flash drives, flash memory, optical media, magnetic media, or any other type of output. It should also be noted that the invention may be implemented on any number of different types of computing devices, e.g., personal computers, laptop computers, notebook computers, netbook computers, handheld computers, personal digital assistants, mobile phones, smartphones, tablet computers, and also on devices specifically designed for these purposes. In one implementation, a user of a smartphone or Wi-Fi—connected device downloads a copy of the application to their device from a server using a wireless Internet connection. An appropriate authentication procedure and secure transaction process may provide for payment to be made to the seller. The application may download over the mobile connection, or over the Wi-Fi or other wireless network connection. The application may then be run by the user. Such a networked system may provide a suitable computing environment for an implementation in which a plurality of users provide separate inputs to the system and method. In the below system where patient monitoring is contemplated, the plural inputs may allow plural users to input relevant data at the same time.

While the invention herein disclosed is capable of obtaining the objects hereinbefore stated, it is to be understood that this disclosure is merely illustrative of the presently preferred embodiments of the invention and that no limitations are intended other than as described in the appended claims. For example, the invention can be used in a wide variety of settings.

1. A system for monitoring a physiologic parameter, comprising:
a. a conformal ultrasonic transducer array coupled to a flexible substrate;
b. an analog front end circuit coupled to the flexible substrate and further coupled to the conformal ultrasonic transducer array, the analog front end circuit configured to generate ultrasonic acoustic waves and receive reflected ultrasonic acoustic waves;
c. a digital circuit coupled to the flexible substrate and further coupled to the analog front end circuit, the digital circuit configured to at least:
i. control the analog front end circuit at least in its generation of ultrasonic acoustic waves;
ii. transmit an indication of the received reflected ultrasonic acoustic waves to an external computing environment.

2. The system of claim 1, further comprising the external computing environment.

3. The system of claim 1, wherein the external computing environment is configured to generate and display an indication of the monitored organ function.

4. The system of claim 1, wherein the external computing environment is configured to measure a shift, the shift in the time domain, in a detected peak of the received reflected acoustic wave, the shift due to movement of an organ or tissue, and wherein the displayed indication of the monitored physiologic parameter is based on the measured shift.

5. The system of claim 4, wherein recognition of the shift is based at least in part on a step of machine learning.

6. The system of claim 5, wherein the displayed indication is based on a step of machine learning, the machine learning associating the shift with the monitored physiologic parameter.

7. The system of claim 1, wherein the analog front end is further configured to steer or direct the generated ultrasonic acoustic waves toward an organ, tissue, or location of interest, the steering or directing by beamforming.

8. The system of claim 7, wherein the steering includes dynamically adjusting a time-delay profile of individual transducer activation in the transducer array.

9. The system of claim 1, wherein the flexible substrate is made of polyimide.

10. The system of claim 1, wherein the transducer array includes a piezo-electric array.

11. The device of claim 1, wherein the monitoring physiologic parameter is central blood pressure or COPD.

12. A method for monitoring a physiologic parameter, comprising:
a. determining a location of interest, the location associated with the physiologic parameter to be monitored;
b. transmitting ultrasonic acoustic waves toward the location of interest;
c. receiving reflected ultrasonic acoustic waves from the location of interest;
d. transmitting an indication of the received reflected ultrasonic acoustic waves to an external computing environment;
e. receiving the received reflected ultrasonic acoustic waves at the external computing environment;
f. detecting a shift in the time domain of the received reflected ultrasonic acoustic wave;
g. determining an indication of the monitored physiologic parameter based at least in part on the shift; and
h. displaying the indication of the monitored physiologic parameter;
i. wherein at least the transmitting and receiving reflected ultrasonic acoustic waves, and the transmitting an indication, are performed by components within an integrated wearable device.

13. The method of claim 11, wherein the monitored physiologic parameter is central blood pressure.

14. The method of claim 11, wherein the transmitting ultrasonic acoustic waves toward the location of interest includes performing a step of steering the ultrasonic acoustic waves toward the location of interest.

15. The method of claim 14, wherein the steering includes dynamically adjusting a time-delay profile of individual transducer activation in the transducer array.

16. The method of claim 11, wherein the transmitting and receiving ultrasonic acoustic waves are performed at least in part by a piezo-electric array.

17. The method of claim 11, wherein the detecting a shift of the received reflected ultrasonic acoustic 16 wave, the shift in a peak in the time domain, includes a step of recognizing the shift using machine learning.

18. The method of claim 11, wherein the determining an indication of the monitored physiologic parameter based at least in part on the shift includes a step of associating the shift with the physiologic parameter using machine learning.

19. The method of claim 16, wherein the machine learning is learned on a training set of ultrasound data.
